(19) United States
(12) Patent Application Publication
(10) Pub. No.: US 2005/0153930 A1
(43) Pub. Date: Jul. 14, 2005

(54) Inhibition of a novel calcium injury current that forms in nuerons during injury prevents neuronal cell death

(75) Inventor: Robert J. DeLorenzo, Richmond, VA (US)

Correspondence Address: OBLON, SPIVAK, MCCLELLAND, MAIER & NEUSTADT, P.C., 1940 DUKE STREET, ALEXANDRIA, VA, 22314, US

(73) Assignee: Robert J. DeLorenzo, Richmond, VA (US)

(21) Appl. No.: 11/008,258

(22) Filed: Dec. 10, 2004

Related U.S. Application Data
(63) Continuation of application No. 09/817,949, filed on Mar. 27, 2001, now abandoned.
(60) Provisional application No. 60/192,994, filed on Mar. 28, 2000.

(51) Int. Cl. A61K 31/727; A61K 31/375; A61K 31/366; A61K 31/28; A61K 31/573

(52) U.S. Cl. 514/56; 514/457; 514/474; 514/492; 514/763; 514/176; 514/179; 514/440; 514/458

(57) ABSTRACT

Calcium channels which form in neuronal membranes in response to injury are disclosed. Methods and compositions for blocking this injury induced calcium channel and preventing further injury to neuronal cells and/or neuronal cell death are disclosed. The compositions and methods disclosed may be used to alleviate acute or chronic injury to neuronal cells.

### FIELD OF THE INVENTION

The present invention relates to compositions and methods for therapeutic treatment of neuronal cells against the influx of calcium induced by cellular injury or disease for the prevention or reduction of neuronal cell death due to acute and chronic injuries to nervous tissue.

### BACKGROUND OF THE INVENTION

Brain or nervous tissue injuries from trauma and cerebral vascular accidents (strokes) represent leading causes of death and morbidity in the United States and around the world. The brain and nervous system are the most vulnerable organs in the body to acute injury and are especially vulnerable to decreased blood or oxygen supplies. In addition the nervous system is especially vulnerable to chronic injuries and aging. Because of the importance in protecting the brain and nervous system following injury or lack of oxygen or blood supply, considerable research has been directed toward developing therapeutic strategies to protect the brain and other components of the nervous system. This research has established that elevation in intra neuronal calcium following acute, excitotoxic, aging, and chronic injuries to nervous tissue is a major cause of irreversible neuronal cell death.

A number of calcium channels are known which permit calcium transport across biological membranes. Strategies to protect the neuronal cells against increased calcium have focused on blocking calcium entry through known calcium channels. However, conventional calcium channel blocking agents have been shown to be effective only if given before or immediately during the initial injury. Research has shown that the treatment must be given within 5-10 minutes of the initial injury to be effective. Araki, T., H. Kato and K. Kogure, Acta Neurol. Scand 80:548-3 1989; Berridge, M. J., Nature 361:315-325, 1993; Kogure, K. and H. Kato, In: Stroke: Pathophysiology, Diagnosis and Management, New York: Churchill Livingston, 1992, p.69-101. Further, many of the positive test results have been reported in animal trials and use of a number of these calcium channel blocking agents in man with head trauma and stroke has not appeared to be effective. These known calcium channel inhibitors have not been effective in treating injuries to the nervous system.

Currently available drugs are not effective in lowering intra cellular calcium levels that develop 15 minutes after injury. Recent studies have demonstrated a role for prolonged calcium elevation following brain injury as a cause of neuronal cell death. Choi, D. W. J. Neurosci 7:369-379, 1987; Choi, D. W. J. Neurobiol. 23:1261-1276, 1992; Eimerl, S. and M. Schramm, J. Neurochem. 62:1223-1226, 1994; Hyre, K., S. D. Handran, S. M. Rothman, and M. P. Goldberg, J. Neuro. Sci 17:6669-6677, 1997. After 10 minutes of excitotoxic brain injury, hippocampal and cortical neurons lose their ability to excrete calcium and maintain a high calcium level for prolonged periods of time. DeLorenzo, R. J. and D. D. J. Limbrick, Adv. Neurol. 71:37-46, 1996; Limbrick, D. D. J., S. B. Churn, S. Sombati, and R. J. DeLorenzo, Brain Res. 690:145-156, 1995. This prolonged elevation in calcium sets in motion a cascade of molecular events that ultimately result in neuronal cell death. DeLorenzo, R. J. and D. D. J. Limbrick, Adv. Neurol. 71:37-46, 1996; Limbrick, D. D. J., S. B. Chum, S. Sombati, and R. J. DeLorenzo, Brain Res. 690:145-156, 1995; Coulter, D. A., S. Sombati and R. J. DeLorenzo, J. Neurophysiol. 68:362-373, 1992; Dubinsky, J. M., J. Neurosci. 13:623-631, 1993. It has been shown that in association with this irreversible calcium triggered cascade leading to neuronal cell death, the cell maintains an extended neuronal depolarization (END) that also contributes to the chronic elevation in intracellular calcium. Coulter, D. A., S. Sombati, and R. J. DeLorenzo, J. Neurophysiol. 68:362-373, 1992; Sombati, S., D. A. Coulter, and R. J. DeLorenzo, Brain Res. 566:316-319, 1991. Both elevated intracellular calcium and extended neuronal depolarization occur following excitotoxic and anoxic brain injury and these processes are not inhibited by known calcium channel inhibitors or other effective treatments.

Thus, a need exists for a therapeutic treatment to limit calcium influx into neuronal cells following acute stroke, vascular accidents, trauma, cardiac arrest, metabolic coma, aging, chronic neuronal injuries or other types of injury to nervous tissue. There is further need for a treatment effective in preventing or reducing neuronal cell death from injury that may be effectively administered for up to several hours following acute injury or on a chronic basis during the time frame in which patients routinely receive medical attention and treatment following these injuries.

### SUMMARY

This invention offers a new therapeutic approach to the protection of the brain following brain and nervous tissue injury. More particularly it is directed to the therapeutic treatment of neuronal cells to prevent calcium influx following both acute and chronic injury to neuronal tissue from causes such as cerebral brain injury, anoxia, vascular accidents, or as a result of chronic injuries due to degenerative disease, aging, or sub acute conditions.

The present invention is based on inventor's discovery of a new calcium channel that is produced in a neuronal membrane in response to both acute and chronic injury. This new calcium channel has been identified as the injury induced calcium channel (IICC). This novel injury induced calcium channel opens after an injury and remains open allowing calcium to rush into the cell.

The present invention includes a method of limiting calcium influx into injured neuronal cells. The method comprises administering to a patient having injured neuronal cells, an effective amount of a blocking agent comprising a composition which is effective in blocking or inhibiting injury induced calcium channels.

The invention includes a method for protecting neuronal cells from calcium influx. The method comprises administering to a patient an effective amount of blocking agent, the blocking agent comprising a composition effective in blocking injury induced calcium channels.

The invention includes a composition for limiting calcium influx into acutely or chronically injured neuronal cells, the composition comprising an amount of a gadolinium compound effective in blocking injury induced calcium channels and an antioxidant.

The invention includes a composition for limiting calcium influx into acutely or chronically injured neuronal cells, the composition comprising an amount of a gadolinium compound effective in blocking injury induced calcium channels and an anticoagulant.

The invention further includes a composition for limiting calcium influx into acutely or chronically injured neuronal cells, the composition comprising an amount of a gadolinium compound effective in blocking injury induced calcium channels, and antioxidant and an anticoagulant together.

### BRIEF DESCRIPTION OF THE DRAWINGS

The present invention can be more fully understood by reading the following detailed description of the presently preferred embodiments together with the accompanying drawings, in which like reference indicators are used to designate like elements and in which:

FIG. 1 shows the effect of 1 minute (A) and 10 minute (B) glutamate exposures (100 uM glutamate and 10 uM glycine) in the presence (black) or absence (white) of extra cellular calcium on the restoration of baseline intra cellular calcium levels in hippocampal neurons in culture. Glutamate exposure caused neurons to reach peak intra cellular calcium levels of 2.4 uM. Following removal of glutamate (time, 0), baseline intra cellular calcium levels were reached within 20 minutes after the 1 minute (A) glutamate exposure after the 10 minute (B) glutamate exposure and did not return to baseline calcium levels. Removal of extra cellular calcium following glutamate exposure resulted in a more rapid return to baseline calcium levels following both the 1 minute (A) and 10 minute (B) glutamate exposures. The curves represent the average intra cellular calcium levels for 21 neurons for presence of extra cellular calcium and 11 neurons for absence of extra cellular calcium. Intra cellular calcium levels [Ca²⁺]ᵢ are expressed as a ratio. A value of 1 represents 2.5 uM calcium, using Indo-1 as a calcium indicator.

FIG. 2 shows extended neuronal depolarization (END) in a hippocampal neuron in response to excitotoxic glutamate exposure and the effects of: A. Control solution following END. B. Zero sodium (0 Na⁺) solution following END. C. Zero sodium and zero calcium (0 Na⁺/0 Ca²⁺) following END and D. Zero calcium (0 Ca²⁺) following END. Following the induction of END by 10 minutes of exposure to 100 uM glutamate and 10 uM glycine, the cells were washed and placed in control (A) or experimental solutions (B-D).

FIG. 3 shows membrane potential following excitotoxic glutamate exposure after 30 minutes of treatment with control (Ctrl), zero sodium and choline chloride (0 Na⁺, chol), zero sodium and NMDA (0 Na⁺, NMDA), zero calcium (0 Ca²⁺), and zero sodium and zero calcium (0Ca²⁺, 0Na⁺) . The data give the means ±SEM for determinations. *p<0.01. The figure demonstrates that zero calcium, but not zero sodium in the external wash solution could reverse END.

FIG. 4 shows the effect of gadolinium chloride in reversing END. The data represent the means ±SEM for n=11.*p<0.01.

FIG. 5 shows Gadolinium was as effective as zero calcium in reversing END. The data give the means ±SEM for n=8.*p<0.01.

FIG. 6 shows effects of zinc chloride, bepridil and SKF-96365 on END. The data give the means ±SEM for n=6. Inhibition of voltage gated calcium channels with 5 uM Zn C12 did not prevent END. Bepridil, an inhibitor of the Na/Ca²⁺ exchanger did not block END. Inhibition of the calcium release activated channels by SKF-96365 had no effect on END.

FIG. 7 shows current/voltage relationships at voltage steps from −90 to +60 mV for control (▪-▪) and END (●-●). The data give the means ±SEM for n=7. The slope current in the control condition is 250 pico siemens. The slope current in END was 50 nano siemens.

FIG. 8 shows current/voltage relationships as voltage steps from −90 to +60 mV for control (▪-▪) and END in the absence (●-●) or presence of gadolinium chloride (Δ-Δ). The data represent the means and SEM for n=6. The data demonstrate that gadolinium returns the neuron from END to baseline (control) conditions.

### DETAILED DESCRIPTION OF THE INVENTION

The present invention includes the discovery, characterization, and inhibition of the novel calcium injury currents that develop during the initial stages of acute and chronic brain injury, aging injury and injury to neuronal tissues. Hereinafter the term injury is taken to mean any one of acute and chronic brain injury, aging injury and injury to neuronal tissues or any combination of these injuries. The calcium injury current is associated with a previously unknown Injury Induced Calcium Channel (IICC) that forms in neuronal cells following acute and chronic cellular injury. The IICCs have unique properties that are different from previously known and classified calcium channels and are selective for the calcium ion. These IICCs account for the majority of cation movement during extended neuronal depolarization following neuronal injury. Once IICCs are opened, the influx of calcium into the cell is such that it is impossible for the normal calcium homeostatic mechanisms to restore base line calcium levels and the neuron remains depolarized by the significant influx of calcium. The opening of IICC channels and the resultant relentless influx of calcium through this channel plays a major role in triggering neuronal cell death. Prior to the discovery of the IICCs , no explanation for the prolonged elevation in intracellular calcium that occurs during extended neuronal depolarization was available and no known calcium channel blockers had any effect in treating this condition.

The invention provides methods and compositions for blocking calcium transport though the IICCs. Blockage or inhibition of the IICCs during the first few hours after injury prevents the overload of intracellular calcium that ultimately results in neuronal cell death to apoptosis or necrosis. Typically, the window for treatment with these compositions and methods is two hours or longer, providing an extended window of opportunity to administer these treatments. IICC specific blocking compounds prevent calcium entry and allow the cells to restore resting calcium levels which limits neuronal death and prevents acute and chronic brain and nervous tissue injury.

IICCs develop during acute, aging and chronic injuries to the neurons. The IICCs have been distinguished from other previously characterized calcium channels by demonstrating that agents that inhibit the flow of calcium through known channels have no effect on the Injury Induced Calcium Channel. Known channels tested included the voltage gated calcium channels L, T, N, and P. Known inhibitors of voltage gated calcium channels L, T, N, and P had no effect on the IICC. In addition, inhibition of calcium entry through NMDA, kainate and AMPA mediated glutamate activated channels had no effect on reducing calcium transport through the injury induced calcium channel. Blockage of ryanodine and IP3 mediated calcium release from the endoplasmic reticulum also did not block the calcium accumulation in the neuron through the injury induced calcium channel. Further, inhibition of the calcium leak and stretch currents and modulation of calcium uptake into the endoplasmic reticulum did not block calcium entry through the IICC. These and additional properties of the injury induced calcium channel discussed herein further differentiate the IICC from previously known calcium channels and calcium uptake and sequestration systems.

The fact that the IICCs are different from all other previously described calcium channels and are not inhibited by agents that block other channels explain why inhibition of known calcium channels has not proven to be effective in treating brain injury. The known agents are effective on physiological calcium channels and systems that function in the normal neuron while the IICCs are associated with the injured neuron. Once the injury produces these new injury induced calcium channels, none of the previously characterized calcium blocking agents can block the IICCs and limit the entry of calcium into the cell after injury.

The present invention provides methods and compositions that modulate or regulate acute or chronic forms of brain, spinal cord or nerve injury and neuronal cell injury by inhibiting the IICCs that develop during injury. These IICCs permit the abnormal accumulation of intracellular calcium. Calcium transport through these channels cannot be prevented by the use of conventional calcium channel inhibitors. Both acute and chronic forms of injury to the nervous system have been shown to ultimately cause increased glutamate release and the abnormal accumulation of intracellular calcium. This abnormal accumulation of glutamate is the trigger common to both acute and chronic injury to the nervous system and glutamate is the major excitatory neuronal transmitter that leads to the production of IICCs discovered in the present investigation. Thus, formation of IICCs occurs in response to many chronic and acute injuries no matter what their cause. The development of IICCs is one of the unique properties of nervous tissue that make it so vulnerable to injury. The present invention provides methods and compositions to inhibit calcium influx through IICCs in both acute and chronic brain injury from multiple causes and offers a unique ability to prevent injury due to calcium entry into neuronal cells during the injury process.

Acute central and peripheral nervous system injuries are caused by catastrophic events that produce immediate damage to neurons that can result in cell death in a very rapid time frame. The present invention provides methods and compositions to timely respond to these forms of acute brain injury. Timely administration of compositions will prevent calcium entry through IICCs during and after the acute injury process. Results provided below demonstrate that inhibition of IICCs even up to two hours after the acute injury can save neurons that would have died from the injury if IICCs were not inhibited. Use of conventional calcium channel blockers have no neuroprotective effects under these conditions, since they do not inhibit calcium entry through IICCs, the calcium channel that is described herein.

Chronic forms of brain injury, aging injury, and injury to nervous tissue occur over a prolonged period of time. Since the cause of the injury produces gradual cell death, the formation of IICCs occur over time and eventually increase in abundance such that the accumulation of calcium in the neuron overcomes the cell's ability to restore baseline calcium levels and neuronal death occurs. The methods and compositions of the present invention may be administered periodically over time to be able to protect the neuron by chronically inhibiting IICCs as they develop due to chronic injury.

By acutely or chronically inhibiting IICCs the present invention provides a novel method to prevent and treat a wide variety of acute and chronic forms of nervous system and neuronal cell injury.

A. Medical Conditions That Contribute to IICC Formation.

The most common cause of acute brain, spinal cord or nerve injury is from strokes. Strokes occur when the blood supply to areas of the brain and nervous system is blocked due to several causes including ischemic, thrombotic, or hemorrhage strokes or subarrachnoid hemorrhage. The ultimate injury to neuronal cells produced by lack of blood supply is the production of IICCs and the development of neuronal cell death. Inhibiting calcium influx through IICCs may improve the out come from strokes and may even totally protect the brain following strokes.

Another form of acute brain, spinal cord or nerve injury and neuronal cell death is produced by cardiac arrest, which causes acute cessation of the blood supply to the brain. Cardiac arrest can be caused by heart failure, myocardial infarctions, electric shock, arrhythmias or numerous other cardiac or medical disorders that cause cardiac arrest. A major injury produced by lack of blood supply from cardiac arrest is the production of IICCs in neuronal cells and the development of neuronal cell death. By inhibiting calcium influx through IICCs following cardiac arrest, damage to the nervous system resulting from cardiac arrest may be alleviated.

Another form of acute brain, spinal cord or nerve injury and neuronal cell death is produced by head, spinal cord, or body trauma. Injury to neuronal cells is produced by acute cessation of the blood supply to the brain, decreased oxygenation of the blood, or direct injury to brain or neuronal tissue or to the central nervous system. Traumatic injury to the nervous system can occur from many internal and external causes including gunshot wounds, head or body trauma from any source, automobile accidents, penetrating injuries, injury from tumors, infections of diseases of the central nervous system and any other cause of direct trauma or injury to the nervous system. A major injury produced by trauma initiates the production of IICCs. Thus, by inhibiting IICCs following trauma, the out come from trauma will be improved and the nervous system may be protected.

Another form of acute brain and nervous system injury and neuronal cell death is produced by lack of oxygenation of the blood that produces hypoxia and/or anoxia and deprives the central nervous system of oxygen. Many conditions produce anoxia or hypoxia. Some of these conditions include any condition that blocks the ability of the lungs to oxygenate the blood such as pulmonary arrest, drug overdose, airway obstruction, chemical poisoning that blocks the ability of the body to oxygenate the blood, drowning, smoke inhalation, burns, infections of the lungs, tumors of the lung, acute allergic reactions, acute and chronic pulmonary disease that cause respiratory failure, lack of oxygenation or injuries produced by multiple causes due to anesthesia or surgical intervention, gun shot wounds, wounds due to numerous causes (knives, tools, shrapnel, various weapons), interference with oxygenation due to chemical weapon agents, nerve gas exposure, insecticides, medications or poisons, snakebite, numerous medical conditions that interfere with the function of the lungs and any other conditions that cause respiratory failure and interference with oxygenation of the blood. A major result produced by all these conditions that can cause an inability to oxygenate the blood or injured nervous tissue is the initiation of events that lead to the production of IICCs and the development of neuronal cell death. Inhibiting IICCs following anoxia and/or hypoxia may protect the brain and central nervous system.

Another form of acute brain, spinal cord or nerve injury and neuronal cell death is injury produced by medical and neurological diseases that can cause acute damage to the nervous system. Medical and neurological diseases that commonly cause injury to the nervous system include hypoglycemia, liver failure, renal failure, coma, repeated seizures and status epileptics. A major injury produced by medical diseases that can cause acute damage to the central nervous system is the production of IICCs and the development of neuronal cell death. Inhibiting IICCs during and following acute medical and neurological diseases may protect the nervous system.

Another form of acute brain, spinal cord or nerve injury and neuronal cell death is injury produced by conditions that can develop during medical procedures such as anesthesia and surgery. For example, it has been well established that prolonged or complicated surgical procedures on the heart, abdomen, brain and other tissues can result in injury to the nervous system, even when the surgery and anesthesia seemed to proceed without apparent complications. Many injuries can occur to the nervous system during surgery and anesthesia that impair the oxygenation of the blood, lower perfusion of the nervous system, or cause abnormal metabolic or toxic effects. Injury produced during anesthesia and surgery can cause acute damage to the nervous system, resulting in increased glutamate production and the production of IICCs and the development of neuronal cell death. By inhibiting IICCs before, during and following anesthesia and surgery that cause damage to the nervous system it may be possible to protect the nervous system during anesthesia and surgery.

Chronic injury to the nervous system is also a major cause of morbidity and mortality. Chronic injury to the central and peripheral nervous system can also cause severe acute and chronic pain, disability, and have a devastating economic effect of society due to the prolonged nature of these conditions. A major feature attributed to chronic neuronal diseases is that they can gradually alter intracellular calcium levels in the neuron and ultimately cause cell death. Chronic injury to the neuron may result from production of IICCs that accumulate in abundance over time in the neuronal membrane. Thus, rather than a rapid overwhelming development of IICCs after acute injury, the chronic nervous system conditions may produce a gradual accumulation of these injury induced calcium channels that eventually reach a critical level to produce an irreversible condition where cells can no longer restore intracellular calcium levels to baseline conditions. When the IICCs reach this critical abundance in the neuronal membrane, the neuron dies. The present invention provides methods and compositions that modulate or regulate the production or formation of IICCs in the neuronal membrane and ultimately delay or prevent the development of neuronal cell death due to these chronic conditions.

Major causes of chronic brain injury and neuronal cell death are injuries produced by Huntington's Chorea, Alzheimer's disease or any of the degenerative dementias such as Pick's disease, senile dementia, and multi infarct dementia, Parkinson's disease, or any of the combined system degenerative diseases associated with Parkinson's ideas such as Progressive Supranuclear Palsy, degenerative cerebellar and brain stem diseases and Shy Dragers disease. These neurodegenerative dementing and disabling conditions ultimately lead to a gradual increase in the production of IICCs and the development of neuronal cell death. By chronically inhibiting IICCs, the chronic injury to the nervous system produced by these conditions and the many other neurodegenerative neuronal diseases may be alleviated.

Another cause of chronic brain injury and neuronal cell death is injury produced by spinocerebellar degenerative disease degenerative diseases of the spinal cord or other parts of the nervous system, such as Friedreich's ataxia, neuropathy, polyneuropathy, and cerebellar degeneration. By chronically inhibiting IICCs, the out come from the chronic injuries to the nervous system produced by these diseases and the many other neurodegenerative neuronal diseases associated with brain stem, spinal cord and peripheral nerve tissue degenerative conditions may be improved.

Another cause of chronic brain injury and neuronal cell death is injury produced by several conditions that produce chronic injury to the nervous system believed to be due to glutamate neurotoxicity, including amyotrophic lateral sclerosis, Parkinsonian-dementia, olivopontocerebellar atrophy, many other rare forms of neurodegenerative diseases, recurrent seizures associated with epilepsy and the prolonged seizures of status epilepticus. These conditions ultimately lead to a gradual increase in the production of IICCs and the development of neuronal cell death. By chronically inhibiting IICCs, damage to the nervous system and neuronal cells may be alleviated.

Another cause of chronic brain injury and neuronal cell death is injury produced by the aging process. There is growing evidence that the aging process produces neurons and cells that leak calcium and have difficulty handling intracellular calcium. Hence, the aging process is associated with chronic accumulation of IICCs in the neuronal membrane and the result is the alteration of normal functioning and ultimately cell death. Aging processes that ultimately lead to a gradual increase in the production of IICCs and the development of neuronal cell death may be mediated chronically by inhibiting IICCs.

B. Experimental Results

The present invention includes the identification of a previously unknown calcium channel and identification of methods and compositions for blocking calcium flow through the channel. The experimental work that underlies the invention has three principal components. These include (i) experimentally creating acutely and chronically injured neurons and establishing procedures for characterizing calcium movement and extended neuronal depolarization (END), (ii) characterization of the novel calcium channel and (iii) identification of compositions which may block calcium transport through the novel calcium channel and prevent acute, aging, and chronic neuronal cell death.

(i) Generating and Characterizing Injured Neurons.

Excitotoxic Glutamate Exposure In Hippocampal Neurons.

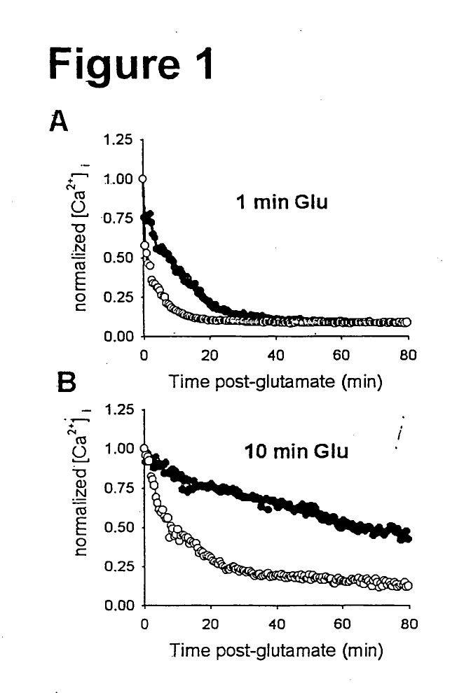

The exposure of hippocampal neurons to excitotoxic glutamate produces a substantial elevation in intra cellular calcium. Both acute and chronic (multiple minor glutamate exposures) injuries can be produced in young and old neuronal populations with these models of injury. In addition, very old neurons die and can be evaluated at their demise to determine the role of calcium in IICCs in this process. These neuronal systems model the physiological increase in glutamate that triggers injured neurons to have sustained elevations of intra cellular calcium in acute, aging or chronic neuronal injuries. Intra cellular calcium was measured in hippocampal neurons using a standardized ratio metric calcium indicator Indo 1 and the ACAS Ultima confocal scanning laser cytometer. In addition both young and old neurons were used to evaluate the role of the treatments and compositions discovered herein on the aging process. Neurons that were exposed to multiple chronic minor glutamate injures were used as a model of chronic injury over time, as is seen in many of the chronic conditions described herein. This chronic model was used to evaluate the effects of the treatments and compositions described herein on chronic glutamate injury. Resting intra cellular calcium levels in hippocampal neurons in culture ranged between 75-175 nM. Neurons were exposed to 100 uM glutamate and 10 uM glycine for one minute as shown in FIG. 1A. This exposure caused an increase in intracellular calcium to approximately 2.4 uM. The one-minute glutamate exposure caused intra cellular calcium to remain elevated beyond the glutamate for a brief time and calcium levels then returned to baseline within 20 minutes.

In contrast to the one-minute exposure, neurons exposed to 10 minutes of glutamate treatment developed an intra cellular calcium peak of approximately 2.4 uM calcium, but this calcium level never returned to baseline level and the cells maintained an elevated intra cellular calcium level as shown in FIG. 1B. Calcium remained above 1 uM for as long as two hours following the initial glutamate therapy. Exposure of glutamate for 15 minutes, 20 minutes, and 30 minutes produced comparable results with elevated calcium reaching peak levels of approximately 2-3 uM. Indo-1 is a high affinity calcium indicator and saturates at the 2 uM level. To better evaluate the actual calcium concentration the calcium indicator Fura-FF was used which has a much lower affinity for calcium, but is more useful than Indo-1 in evaluating calcium levels above 3 uM. Using the Fura-FF indicator, the absolute calcium concentration produced during the injury was 20-40 uM. The intra cellular calcium level remained elevated with both indicators essentially indefinitely following excitotoxic glutamate exposures with monitoring for up to four hours. This prolonged elevation in intra cellular calcium has been implicated in triggering a cascade of molecular events that eventually kill the neuron. DeLorenzo, R. J. and D. D. J. Limbrick, Adv. Neurol. 71:37-46, 1996; Limbrick, D. D. J., S. B. Churn, S. Sombati, and R. J. DeLorenzo, Brain Res. 690:145-156, 1995.

Previous research has demonstrated that neurons that can't restore resting calcium levels to base line levels following glutamate exposure go on to develop delayed neuronal cell death. DeLorenzo, R. J. and D. D. J. Limbrick, Adv. Neurol. 71:37-46, 1996; Limbrick, D. D. J., S. B. Churn, S. Sombati, and R. J. DeLorenzo, Brain Res. 690:145-156, 1995. Thus, the inability to return intra cellular calcium concentration to baseline levels is a marker for neurons that will die. Neurons exposed to glutamate for up to approximately eight minutes were still able to restore calcium to baseline. DeLorenzo, R. J. and D. D. J. Limbrick, Adv. Neurol. 71:37-46, 1996; Limbrick, D. D. J., S. B. Churn, S. Sombati, and R. J. DeLorenzo, Brain Res. 690:145-156, 1995. The sensitivity to glutamate time exposure varied somewhat from culture to culture, but there is a clear distinction between the excitotoxic exposures that resulted in delayed neuronal cell death, as characterized by the 10-30 minute exposures and exposures between one and eight minutes that usually resulted in a majority of neurons returning to baseline calcium levels and not going on to die. This elevation in intra cellular calcium correlated in a highly statistically significant fashion with the development of cell death.

Excitotoxic Neuronal Injury and Neuronal Depolarization.

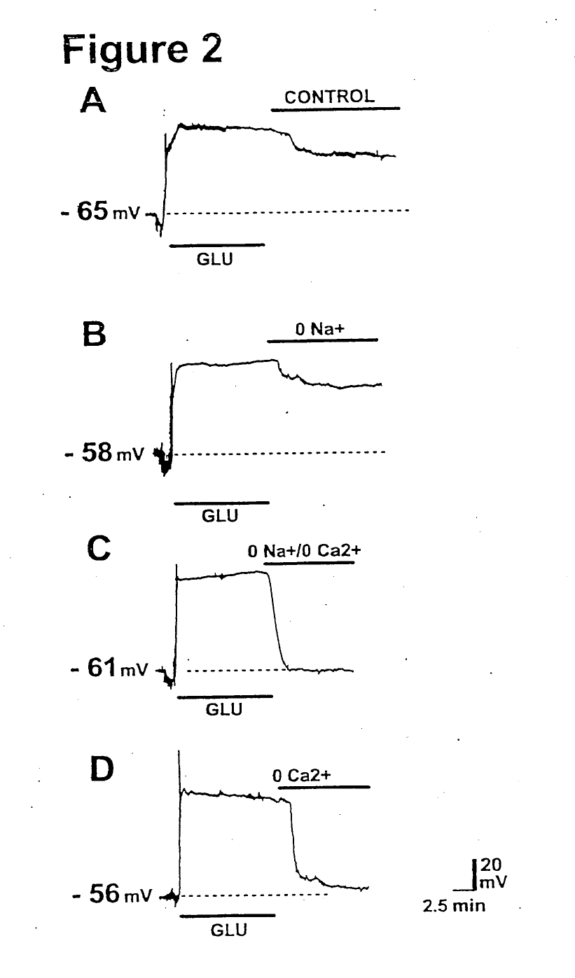

Experiments were conducted to evaluate the effects of excitotoxic glutamate exposure for 10 minutes on neuronal membrane potential. Neuronal resting membrane potential was recorded before, during, and after glutamate exposure as shown in FIG. 2. Neurons had baseline resting potentials between ±70 and −55 mV. Following exposure to 100 μM glutamate and 10 μM glycine for one minute, neurons depolarized to a resting potential of approximately 2-5 mV and rapidly returned to −65 mV following the end of the one-minute exposure. The return to baseline of resting potential was much more rapid than the return to baseline calcium levels.

As shown in FIG. 2A, exposure of hippocampal neurons to 100 uM glutamate and 10 uM glycine for 10 minutes caused the cells to become depolarized, reaching levels of +5 mV. However, neurons remained depolarized between −15 and 0 mV indefinitely e.g. for the entire period of measurement which was as long as two hours after the removal of glutamate in some experiments. These results demonstrated that neurons exposed to excitotoxic glutamate exposures, not only could not restore baseline calcium levels, but also could not restore resting membrane potential to normal values. Inability to restore resting membrane potential levels was called extended neuronal depolarization (END). Coulter, D. A., S. Sombati, and R. J. DeLorenzo, J. Neurophysiol. 68:362-373, 1992.

Studies on old neurons demonstrated that aging gradually increased the sensitivity of the neuron to injury and the ability to develop END. As neurons became very old they developed END and died. In comparison to young neurons, older neurons developed END at much lower levels of glutamate injury. In addition the production of multiple minor glutamate injuries with shorter exposure times or lower concentrations of glutamate also eventually produced END, indicating that accumulation of multiple chronic injuries can eventually produce END.

Calcium Influx Following Excitotoxicity Neuronal Injury.

Calcium imaging was used to investigate the influence of extra cellular calcium on the protracted calcium elevation in the period following excitotoxic glutamate exposure as shown in FIG. 1. Indo-1 was used as a calcium indicator and cells were exposed to excitotoxic glutamate conditions. Following the development of prolonged elevations in intra cellular calcium during the glutamate exposure for 10 minutes, cells were exposed to normal wash solution or to a solution containing zero calcium. The cells exposed to the normal wash served as control and manifested the prolonged elevation in intra cellular calcium. However, the calcium level in the zero calcium wash treated cells rapidly returned to baseline conditions. Thus, the chronic elevation in intra cellular calcium produced by excitotoxic glutamate exposure was influenced by entry of extra cellular calcium, since removal of the extra cellular calcium during this elevated phase rapidly lowered the elevated intra cellular calcium.

Following the elevation of calcium from a 10 minute glutamate exposure, preparations washed with zero calcium restored to baseline calcium levels within 30 minutes as shown in FIG. 1B. All of the neurons that were washed with normal controlled solutions did not return to standard baseline calcium levels. This data indicated that extra cellular calcium entry was contributing to the prolonged elevation of intra cellular calcium observed after excitotoxic glutamate exposure. This result provides direct evidence that entry of calcium was contributing to the prolonged elevation of intra cellular calcium.

Studies on old neurons demonstrated that aging gradually increased the sensitivity of the neuron to injury and as the neuron aged, it developed increased calcium influx and a prolonged elevation of intra cellular calcium levels. As the neurons became very old they developed prolonged elevation of intra cellular calcium levels that resulted in cell death. In comparison to younger neurons, older neurons developed calcium influx and the prolonged elevations of intra cellular calcium levels at much lower levels of glutamate injury.

Results also demonstrated that minor chronic injuries from glutamate exposure that added up eventually to a significant injury over time caused elevated calcium levels and neuronal cell death. Production of multiple minor glutamate injuries with shorter exposure times or lower concentrations of glutamate also eventually produced increased calcium influx and prolonged elevations of intra cellular calcium levels. When chronic injuries eventually caused increased calcium levels in neurons, the neurons died. These results indicated that accumulation of multiple chronic injuries could eventually produce increased calcium influx and the prolonged elevations of intra cellular calcium levels.

Calcium Entry Contributes To Extended Neuronal Depolarization.

Since the calcium imaging experiments discussed above indicated that elevated intra cellular calcium levels in neurons following excitotoxic injury were being affected by prolonged entry of extra cellular calcium, studies were performed to determine whether the extended neuronal depolarization seen in conjunction with the elevated calcium was also influenced by calcium entry. Following exposure to 100 μM-glutamate for 10 minutes, neurons were then washed with controlled or low-calcium solutions. Cells washed with the normal calcium containing media remained in END as shown in FIG. 2A. However, cells washed with 0-calcium solution rapidly returned their resting membrane potential to baseline levels in the −70 to −50 mV range as shown in FIG. 2D. The same results described above were also obtained in evaluating elevated calcium levels produced by aging and chronic glutamate injuries.

These results demonstrated that entry of extra cellular calcium was a major cation contributing to the extended neuronal depolarization observed after excitotoxic glutamate exposure in acute, aging and chronic injuries, and significantly contributed to the development of END. The above data indicate that calcium entry during END was a major factor contributing to the development of END.

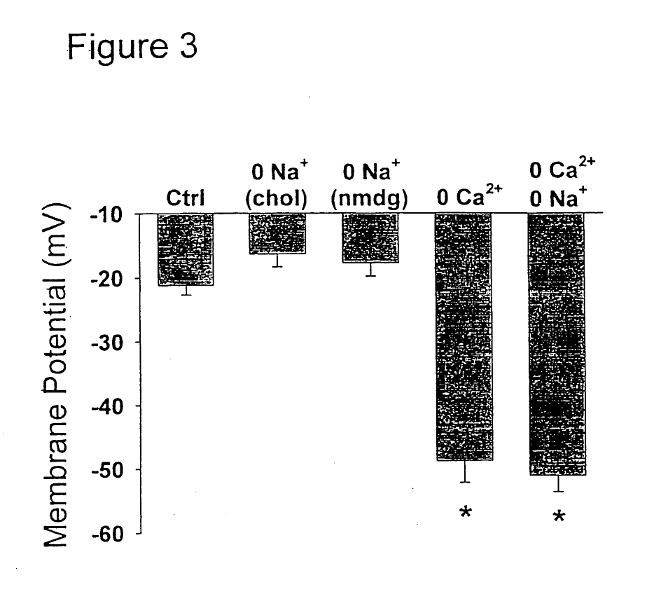

It was important to show that sodium, which is the major cation normally utilized under physiological conditions for depolarizing neurons is not significantly involved in producing END. Following the development of END after 10 minutes of glutamate exposure, neurons were washed with control solutions containing all components in the media except for zero sodium with a sodium substitution with choline chloride or NMDG chloride as shown in FIGS. 2B and 3. Treatment with zero sodium could not restore neurons to resting membrane potential. Neurons went from +5 to approximately −10 mV following exposure and wash with zero sodium solutions. In addition, substitution of sodium with choline chloride or NMDG chloride solutions in similar concentrations did not restore cells to baseline resting potential after 30 minutes. Thus, removal or substitution of sodium as the major extra cellular cation did not significantly affect END, demonstrating that after the development of excitotoxic injury, the prolonged depolarization could not be reversed by removal or replacement of sodium in the extra cellular media.

This is in sharp contrast to the fact that removal of extra cellular calcium was able to reverse END as shown in FIG. 2D and FIG. 3. In addition, removal of sodium and calcium from the wash used after glutamate exposure was also able to restore the neuron to baseline membrane potentials as shown in FIG. 2C and FIG. 3. These results provide evidence that calcium entry and not sodium entry was contributing to the maintenance of END. These results also suggest that a calcium injury current may have formed during the injury process, produced by acute, aging and chronic injuries.

Agent for Blocking Prolonged Elevation Of Intra Cellular Calcium And END.

Since calcium entry contributes to the development of prolonged elevated levels of calcium and END, experiments were conducted to determine whether a composition could be identified that would block the prolonged elevation of intra cellular calcium and END that occur after excitotoxic neuronal cell death, caused by acute, aging or chronic injuries. An ion with a relatively large ionic radius was selected, namely, gadolinium. Gadolinium compounds are used as a contrast agents for magnetic resonance imaging (MRI) studies and thus are safe for use in humans. Experiments were conducted to evaluate the possibility that gadolinium chloride could reverse END in hippocampal neurons in culture.

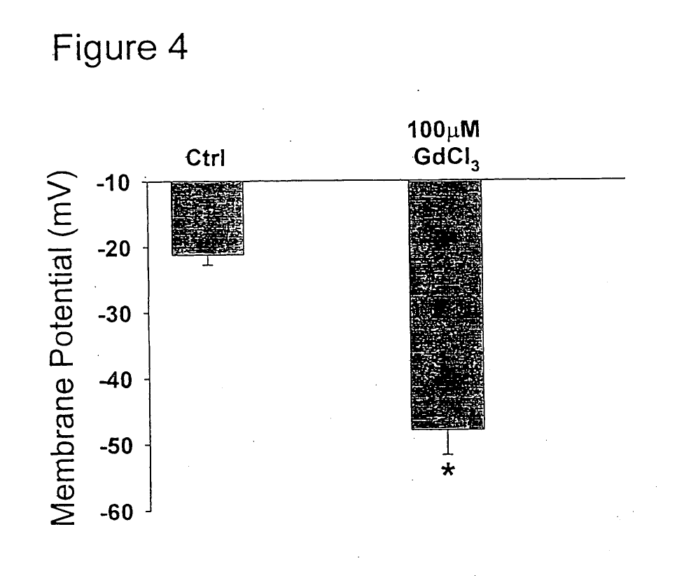

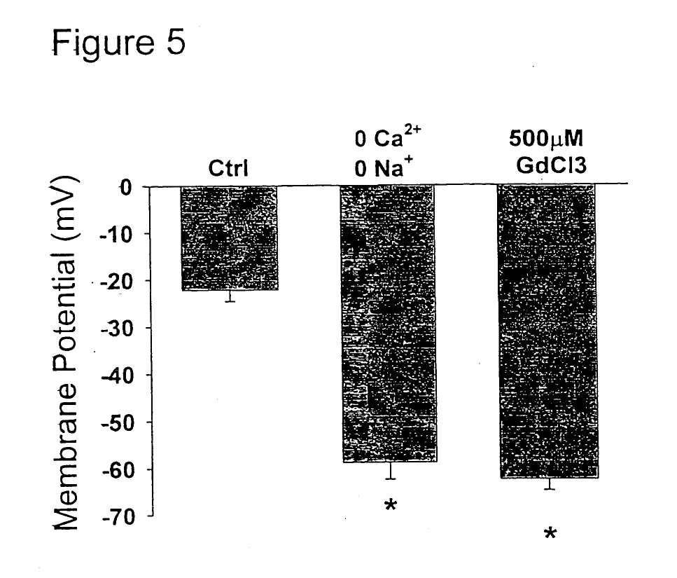

Following exposure to excitotoxic glutamate levels, gadolinium was applied in the wash solution as shown in FIG. 4 and FIG. 5. Control preparations had intra cellular calcium levels with indo-1 that were sustained at approximately 1-2 uM and manifested END for greater than one hour. However, treatment with gadolinium following the glutamate exposure resulted in a much more rapid decrease in intra cellular calcium and END, returning to baseline levels in approximately 30-40 minutes. These results indicate that gadolinium was able to block the extra cellular calcium entering from the bathing solution and thus allowed the neurons to restore resting calcium levels and membrane potential. Gadolinium was able to reverse END as well as zero calcium in the extra cellular wash as shown in FIG. 5. Other forms and compounds of gadolinium were also determined to be effective inhibitors. Thus, gadolinium compounds were determined to be inhibitors of the calcium induced toxicity and the entry of calcium following injury.

Gadolinium blocked calcium entry and the development of END when chronically administered during aging. Gadolinium was effective in significantly increasing the life span of the neurons. In addition, chronic administration of gadolinium blocked calcium entry and accumulation and the development of END in the chronic injury model.

Treatment with gadolinium compounds had the same effect on the prolonged elevation of calcium seen following injury as it did on END. These results demonstrated that both END and prolonged elevation of intra cellular calcium that are affected by extra cellular calcium were blocked by gadolinium. These results provide strong evidence that blockage by gadolinium of the entry of calcium following acute, aging, and chronic neuronal injury can block both END and prolonged elevation of intra cellular calcium and prevent or reduce neuronal cell death.

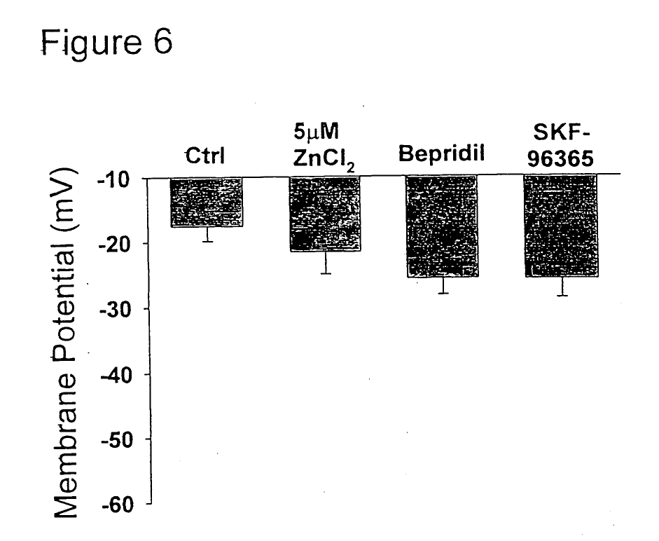

Further attempts were made to reverse END, these included treatment with zinc, bepridil, and SKF-96365. 5 uM zinc, which inhibits voltage gated calcium channels, had no effect in reversing END as shown in FIG. 6. Bepridil, a potent inhibitor of the sodium/calcium exchange pump, had no effect in reversing END as shown in FIG. 6. Further inhibition of the calcium release activated currents with SKF-96365 had no effect on reversing END. These results further suggest that END was maintained by a novel calcium injury current that developed during acute, aging and chronic neuronal injuries.

(ii) Development of A Novel Calcium Current Following Neuronal Injury.

Since removal of extra cellular calcium had a significant effect on both END and prolonged elevation of intra cellular calcium, the possibility that a novel calcium current developed during acute, aging, and chronic neuronal injury was explored.

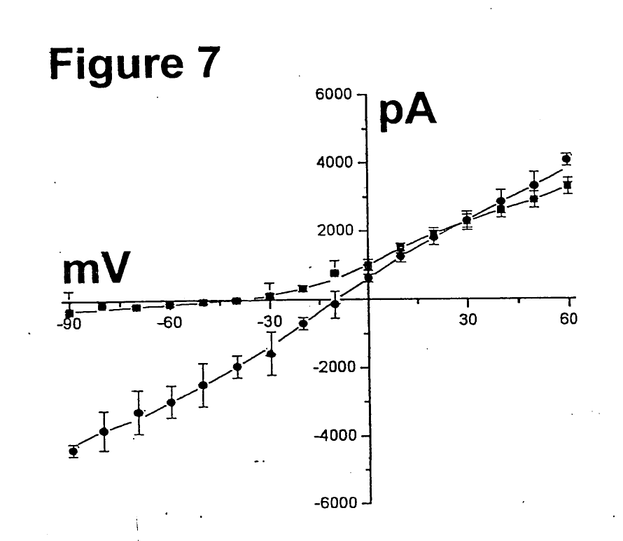

To evaluate the role of the IICC current in the entry of calcium into a neuron following excitotoxic injury, intra cellular recordings were obtained from control neurons and neurons exposed to acute excitotoxic glutamate exposure, aging and chronic glutamate exposures as described above. The current voltage relationships for these conditions were determined at intervals from −90 to +60 mV. FIG. 7 presents the current/voltage relationships for control and END neurons after acute injury. This experiment demonstrates that the slope current for the control neurons was very small (250 pico siemens). However, during END there was a very large slope current of 50 nano siemens. The results confirm the studies with calcium imagining and with membrane potential recordings that suggested that END produced a significant inward injury current. Essentially identical results to those shown in FIG. 7 were observed for the current voltage relationships for aging and chronic glutamate injuries that eventually produced END.

The inward current observed in END was further characterized by removal of calcium from the recording solution. This completely blocked this calcium injury current. Furthermore, the role of calcium in mediating this current was explored by evaluating the effects of increasing and decreasing external calcium concentrations in the development of the magnitude of this current. The current voltage relationship of this current was shifted proportionately by increasing or decreasing calcium. This provides strong evidence of a calcium current. Voltage clamp results provide electrophysiological evidence for the development of a novel calcium current following acute excitotoxic injury. This current has not been described previously and represents a new finding of a biophysical characterization of a channel that develops during neuronal injury, e.g. the IICC.

Identical results to the acute injury were obtained with neurons that developed END as a result of aging and chronic less severe glutamate injuries. The IICCs were found to develop in the neuronal membrane during the aging of the neurons and developed more dramatically in response to glutamate in old neurons compared to young neurons. In addition chronic minor injuries with lower concentrations and durations of glutamate produced a gradual accumulation of these channels over time in the neuronal membrane. These results indicated that IICCs play a role in aging and in the chronic injury preparation and demonstrate that the gradual accumulation of these injury induced calcium channels can eventually produce a high level of these channels that result in the same effect as acute injury, and ultimately cause neuronal cell death.

Characterization of the Injury Induced Calcium Current.

The injury-induced calcium current described above was further characterized to evaluate its identity. Voltage clamp studies were conducted following excitotoxic neuronal injury or on control neurons using various substitution solutions to evaluate the ionic dependence of this calcium injury current. Removal of extra cellular sodium or replacement of extra cellular sodium with choline chloride or NMDG did not diminish the injury current indicating that the injury current was not significantly affected by extra cellular sodium. These results indicate that this new current or channel was selective for calcium over sodium. Identical results were obtained following the injuries produced by aging and chronic glutamate injuries.

In addition, experiments were conducted to evaluate the possible role of the sodium/calcium exchange pump in regulating membrane potential after END induced by acute, aging and chronic glutamate injuries. Experiments conducted in the presence of bepridil had no effect on blocking the observed calcium injury current seen in END. Further pharmacological characterization of this current in the three types of injury was conducted using known inhibitors of voltage gated calcium channels. The effects of nifedipine, conatoxin and cadmium were evaluated. None of these calcium channel blockers had any effect on the calcium leak current.

The effects of glutamate receptor antagonists and channel inhibitors were also evaluated on IICC current in all three types of injury. The effects of CNQX, NBQX, and APV were evaluated. None of the glutamate receptor antagonists had any effect on the magnitude of the IICC currents. The effect of other neuro active molecules, on the magnitude and development of this current was examined. Inhibition of chloride currents and potassium currents had no effect on the IICC current. Inhibition of the calcium release affected currents also had no effect on the IICC current.

Based on these studies characterizing the IICC current, the data indicate that this is a novel calcium current that has not been previously described. The previously known and described calcium channel antagonists including both excitatory amino acid activated calcium entry or voltage gated calcium channel blockers had no effect on the IICC current. In addition, the selectivity of the IICCs for calcium and inability to conduct other known ions was demonstrated. These results strongly indicate that this is a unique type of IICC current that develops after acute excitotoxic injury, aging, and chronic less severe excitotoxic injuries.

(iii) Compositions Which Block Calcium Transport by the IICC.

Gadolinium is an Inhibitor of the IICC.

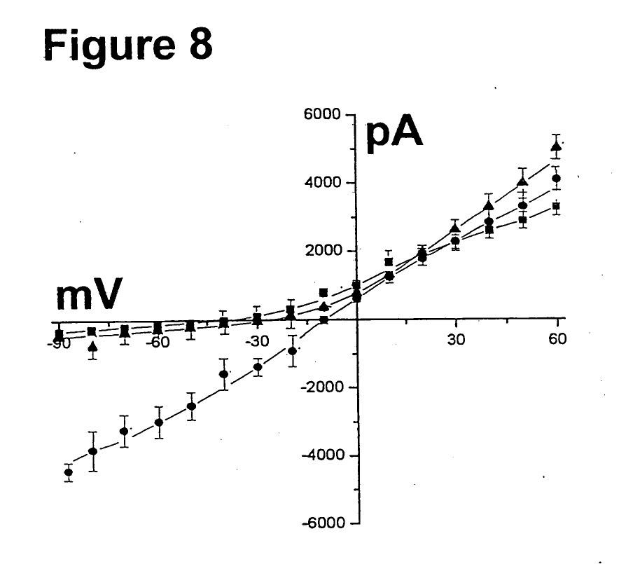

Since gadolinium effectively reversed END and the prolonged elevations in intracellular calcium, studies were initiated to evaluate the effects of gadolinium on the IICC current. Gadolinium effectively inhibited the IICC current and returned the neuron to baseline conditions as shown in FIG. 8. In addition to blocking the IICC currents gadolinium was also effective in reversing END. Gadolinium was also effective in reversing the prolonged elevation of intra cellular calcium following excitotoxic injury and the cell death produced by excitotoxic glutamate exposure.

Gadolinium inhibition of the IICCs resulted in prevention of delayed neuronal cell death. Using gadolinium inhibited the IICC current and prevented the effect of excitotoxic neural injury. Gadolinium was effective in preventing cell death when given up to one hour after excitotoxic injury. Further, neurons that were exposed to excitotoxic injury in the presence gadolinium did not die.

Additionally, gadolinium compounds administered chronically to neurons as they aged, were able to protect aging neurons from the accelerated cell death associated with the aging process. Gadolinium given chronically blocked the development of IICCs and prolonged the life of the neurons and thus was able to block or decrease the effect of aging on neuronal cell loss. Furthermore, chronic exposure to gadolinium during numerous minor chronic glutamate injuries of lower concentration or duration blocked the formation of IICCs and prevented the gradual production of neuronal cell death seen from chronic injury. Thus, gadolinium was effective in blocking or reducing the effects of aging and chronic multiple injuries on causing cell death.

Antioxidants and Anticoagulants Increase the Effectiveness of Gadolinium.

Antioxidants, e.g. vitamin E (alpha-tocopherol), vitamin C (ascorbic acid), methlyprednisolone, alpha-lipoate thioctic acid, 1,2-dithiolane-3-pentanoic acid, 1,2-dithiolane-3 valeric acid, and 6,8-dithiooctanoic acid, ascorbyl palmitate, dilauryl ascorbate, beta-carotene, nizofenone and tirilazad mesylate] and anticoagulants, e.g. heparin, heparin derivatives, low molecular weight heparins and coumarin administered alone or in combination after the 10 minute glutamate exposure had no significant effect on neuronal survival. However, administering either an antioxidant or anticoagulant in combination with gadolinium increased the effectiveness of gadolinium in blocking neuronal injury. The addition of an antioxidant and/or anticoagulant to gadolinium increased the effectiveness of gadolinium in blocking IICCs and in preventing neuronal cell death. Furthermore, the addition of either an antioxidant or anticoagulant to gadolinium extended the time after injury that gadolinium was able to reverse or prevent injury. This combination of an antioxidant or anticoagulant and gadolinium extended the time for reversing injury for up to two hours after acute excitotoxic injury.

Administering gadolinium and both an antioxidant and an anticoagulant agent was effective in reversing injury and preventing cell death when given up to two and a half hours after excitotoxic injury. These findings demonstrate that the combination of either antioxidants or anticoagulants with gadolinium was more effective than gadolinium alone. Furthermore, the combination of both an antioxidant and an anticoagulant with gadolinium was the most effective treatment for reversing or preventing neuronal injury.

Gadolinium plus an antioxidant, gadolinium plus an anticoagulant, and gadolinium plus both an antioxidant and anticoagulant administered chronically to neurons as they aged were able to protect aging neurons more effectively than gadolinium alone from the accelerated cell death associated with the aging process.

Gadolinium plus an antioxidant, gadolinium plus an anticoagulant, and gadolinium plus both an antioxidant and anticoagulant were able to more effectively chronically inhibit the formation of IICCs, and prolonged the life of the neurons and thus decrease the effects of aging due to the development of IICCs and neuronal cell death.

Furthermore, chronic exposure to gadolinium plus an antioxidant, gadolinium plus an anticoagulant, and gadolinium plus both an antioxidant and anticoagulant during numerous minor chronic glutamate injuries of lower concentration or. duration were able to more effectively prevent the gradual production of IICCs and neuronal cell death seen from chronic injury. Thus, gadolinium plus an antioxidant, gadolinium plus an anticoagulant, and gadolinium plus both an antioxidant and anticoagulant were more effective than gadolinium alone in blocking or reducing the effects of aging and chronic multiple injuries on the development of IICCs and in causing cell death.

B. Methods and Compositions for Therapeutic Treatment of Neuronal Cells.

The methods and compositions of this invention alleviated both acute, aging, and chronic injury to neuronal cells by blocking or inhibiting injury induced calcium channels. While the therapeutic compositions are useful for treating acute, aging and chronic neuronal injury, the method of administration may be varied to optimize treatment of an acute or chronic condition. Further, it will be appreciated by one skilled in the art that the therapeutic method and compositions described herein may be used with both human and animal patients and that use of the term patient includes both human and animal.

Acute central and peripheral nervous system injuries are caused by catastrophic events that produce immediate damage to neurons that can result in cell death in a very rapid time frame. Methods for treating acute brain injuries include rapid administration of compositions that prevent calcium entry through IICCs for an acute injury treatment during and immediately after the acute injury process are desirable. Experimental results demonstrate that inhibition of IICCs at least up to two hours after the acute injury can save neurons that would have died from the injury if IICCs were not inhibited. Use of conventional calcium channel blockers have no neuroprotective effects at times beyond 5-10 minutes after injury and also have no affect on blocking IICCs that permit the influx of calcium and elevation of calcium in the neuron after injury.

Chronic forms of brain injury occur over a prolonged period of time. Chronic forms of injury include injuries produced by neuronal aging and multiple minor glutamate injuries that gradually cause the development of IICCs and neuronal cell death. Formation of IICCs occurs gradually, over time in chronic injuries and accumulates in the neuron to eventually overcome the cells ability to restore baseline calcium levels and produce neuronal death. Methods and compositions for treating aging and other chronic forms of injury are preferably administered gradually over time to be able to protect the neuron by chronically inhibiting IICCs as they develop due to chronic injury.

Neuronal injury is a trigger that causes a change in the environment of the neuron with the accumulation of the excitatory neurotransmitter, glutamate, and other agents. It has been discovered in this research that the excessive accumulation of these agents results in the formation of IICCs that can form rapidly in response to acute injury or more gradually in response to aging and other chronic injuries. The IICCs permit calcium influx into neuronal cells yielding abnormal levels of intra cellular calcium, which can cause neuronal cells injury and death. Thus, inhibition of formation of IICCs and calcium influx through IICCs to prevent or reverse injury to neurons is desirable. Conventional calcium channel inhibitors do not affect these IICCs. Thus, the known calcium channel blockers are not effective in preventing neuronal injury once the IICCs develop in response to injury.

The present invention provides compositions that can deliver compounds that block IICCs and prevent the damaging accumulation of calcium in the neuron in both acute and chronic conditions that are currently resistant to standard calcium channel inhibitors and agents. The phrase inhibit IICC, or inhibit IICCs or inhibition of IICCs is taken to mean either the inhibition of the formation of an IICC or inhibition of calcium transported through an IICC. In either case the end result is blockage of entry of calcium into the neuronal cells via an IICC. Acute injuries require the immediate delivery of the blocking agents and chronic injuries and aging necessitate a delivery system delivering blocking agents over time.

It has been discovered that inhibition of IICCs even several hours after injury can be effective in reversing neuronal injury, which provides a significant window of opportunity to treat acute injury to the nervous system by inhibiting IICCs. It has also been unexpectedly discovered that inhibition of IICCs also make the neuron responsive to other neuroprotective agents e.g. antioxidants and anticoagulant agents that have previously not be effective once the IICCs developed. Thus, the combination of neuroprotective agents with an inhibitor of IICCs offers a new and unexpected ability to treat acute, aging and other chronic neuronal injury.

The present invention utilizes the unexpected finding that gadolinium compositions can reverse damaging effects and prevent further damage to the nervous system for hours after an injury has occurred by inhibiting the IICCs that develop from injury and cause neuronal cell death. Inhibition of IICCs represents a new and unexpected method to treat and prevent acute aging and other chronic injuries to the nervous system.

Gadolinium compositions are an exemplary embodiment of compositions that may function as a blocking agent to inhibit IICCs. A blocking agent as used herein is any composition that can inhibit an IICC. The blocking agent may be administered in response to an injury and may be administered for a period up to several hours after the injury in treatment of an acute injury. Alternatively, the blocking agent may be administered in a systematic manner at periodic intervals in the treatment of neuronal cells subject to a chronic condition that may result in neuronal injury. Since aging and other chronic neurological injuries occur over time, the systematic administration of a blocking agent prevents the chronic build up of calcium in neurons that occurs as the final cause of cell death in the various chronic neurological injuries. The use of chronic delivery systems or time-release or sustained release delivery systems may be employed to provide chronic delivery of the blocking agent of this invention. Further, blocking agents may be administered in anticipation of an injury or compromising condition such as prior to a medical procedure or surgery for example.

Antioxidants and anticoagulants alone have no effect in treating neuronal cells with excessive intra cellular calcium. However antioxidants and anticoagulants have been unexpectedly discovered to enhance the efficiency of a blocking agent in treating neuronal cells.

Combinations of the blocking agent and antioxidant and/or anticoagulants may be used depending on the severity of the injury, the age of the patient, and the route of administration. In an exemplary embodiment these combinations include, for example, the administration of gadolinium plus antioxidant agents, gadolinium plus anticoagulant agents, and gadolinium plus antioxidant plus anticoagulant agents. The severity of the injury, the route of administration, the effectiveness of the formulations, and the condition of the patient are considered when determining which combinations may be employed.

In an exemplary embodiment gadolinium compounds are used as blocking agents. Specific formulation depends on the goal of treatment, whether acute or chronic injury to the nervous system is being treated; the route of administration; and the toxicity of the parent compound in a given route of administration. Gadolinium is available as numerous compounds. Because the gadolinium ion is water-soluble it can be administered in the ionic form as a pharmaceutically acceptable salt of an organic or inorganic acid, for example chlorides (GdCl₃, GdCl₃.6H₂O), fluorides (GdF₃), bromides (GdBr₃), iodides (GdI₂, GdI₃), oxides, (Gd₂O₃), sulfides (Gd₂S₃), selenides (GdSe), tellurides (Gd₂Te₃) and nitrides (GdN) may be used. As one skilled in the art will appreciate gadolinium compounds can be used in various formulations and delivery systems, known to one skilled in the art.

Further, gadolinium complexes may be employed. Gadolinium complexes have the prospective of selectively entering the injured nervous tissue due to alterations of the blood brain barrier. Gadolinium chelates formed by chelating gadolinium ions with various different complexing agents. Their production includes the step of the metallation of the complexing agents with gadolinium. Reacting the complexing agents with gadolinium oxide in a heated aqueous medium can develop metallation with gadolinium. Examples of gadolinium chelates include Gd DTPA, Gd DPTA-BMA, Gd DPTA-MMA, Gd-DO3A-butrol, Gd-DO3A-HP, Gd-DOTA meglumine, Gd-BOPTA/Dimeg, Gd HP-DO3A and the like.

The present invention includes the unexpected discovery that administration of a blocking agent such as gadolinium in various combinations with antioxidant, and/or anticoagulant agents produces a more effective neuronal protective effect and extends the time after the injury that the formulation was effective in preventing injury. These antioxidant and/or anticoagulant agents given alone in the absence of a blocking agent had no effect on an injury after it occurred. In addition these agents did not inhibit injury induced calcium influx into neuronal cells and the resulting cell injury and death. However, when combined with a blocking agent the protective effect of the blocking agent was increased significantly over the blocking agent alone when the blocking agent was combined with an antioxidant and/or an anticoagulant.

Antioxidant agents include for example vitamin E (alpha-tocopherol), vitamin C (ascorbic acid), methlyprednisolone, alpha-lipoate (thioctic acid, 1,2-dithiolane-3-pentanoic acid, 1,2-dithiolane-3 valeric acid, and 6,8-dithiooctanoic acid), ascorbyl palmitate, dilauryl ascorbate, beta-carotene, nizofenone and tirilazad mesylate. These compounds are effective in decreasing lipid peroxidation and free radical production. An unexpected discovery of this invention was that these antioxidant agents when combined with the inhibition of IICCs by a blocking agent produced a significant improvement in outcome from neuronal injury that was greater than expected by the blocking agent alone. Another unexpected finding of this invention was that administering an antioxidant with a blocking agent increased the time after the injury that the treatment was effective in preventing or reducing neuronal cell death and injury to the nervous system.

Anticoagulants prevent of reduce the coagulation of blood components and thus reduce or prevent clot formation. Common anticoagulants include heparin, heparin derivatives, low molecular weight heparins and coumarin. Heparin, low molecular weight heparins, and heparin derivatives also offer the additional advantage of acting as an inhibitor of the IP3 receptor activate calcium induced calcium release system in the endoplasmic reticulum. An unexpected discovery of this invention was that anticoagulant agents used in combination with a blocking agent produced a significant improvement in outcome from neuronal injury that was greater than expected by the blocking agent alone. Additionally, administering an anticoagulant agent in combination with the blocking agent unexpectedly increased the time after the injury that the treatment was effective in preventing or reducing neuronal cell death and injury to the nervous system.

The methods and compositions of the present invention utilize the blocking agent alone or in combination with the antioxidant and/or anticoagulant agents as the specific need requires. The formulation of the composition used will depend on numerous factors, for example the condition of the patient, the nature of the neuronal injury, the age and gender of the patient, the preferred method of delivery, the toxicity of the compounds, and the availability of the route of access.

The blocking agent may be administered alone or in various combinations with the antioxidant and/or anticoagulant agents as pharmaceutical formulations. The pharmaceutical formulations may comprise blocking agents alone or blocking agent with at least one of an antioxidant and an anticoagulant. The components of the formulation may be present as a pure compound or may be combined with one or more pharmaceutically acceptable carriers. An acceptable carrier is taken to mean a carrier that is compatible with the components of the formulation and not deleterious to a patient.

The composition of the present invention may be administered by several routes depending on the nature of the injury and whether it is given by acute or chronic routes of administration including intra arterial; intravenous; intrathecal; intraperitoneal; intramuscular; oral; sublingual; buccal; aerosol (topical or inhalant); nasal drops; subcutaneous; eye drops; ear drops; intracranial; topical (both as patches and direct, for use on skin and on internal organs); intracardiac; electrophoretic; suppository; extracorporeal (used in dialysis, perfusion solutions, and dosing blood organs); and intravaginal. The most suitable route will depend on, for example, the safety of administration, the condition of the patient, the disorder of the recipient, and the acute or chronic nature of the condition being treated. Specific kit(s) may be used to provide a safe and rapid method of administering the agent by the most effective route. These kits may be developed for specific conditions causing injury and for various purposes.

The formulations used may conveniently be presented in unit dosage form and may be prepared by any of the many methods that are well known and standard in the art of pharmacy. Typically, a formulation is made by intimately and uniformly bringing into association the blocking agent or a blocking agent in combination with at least one of an antioxidant and an anticoagulant with liquid carriers or finely divided solid carriers, or both, and then, if necessary, shaping the final product into the desired formulation for optimal delivery to the injured neurons.

Formulations for parenteral administration comprise aqueous and non-aqueous sterile injection solutions that may contain buffers, bacteriostats, antioxidants, and solutes, which render the formulation isotonic with the blood of the recipient, and aqueous and non-aqueous sterile suspensions, which may include thickening and suspending agents. Extemporaneous injection solutions and suspensions may be prepared from sterile powders, granules and tablets described below. The formulations used may be developed in unit-dose or multi-dose containers, as sealed ampules, vials or injection bottles, and may be stored as liquid or freeze-dried (lyophilized) condition requiring the addition of sterile liquid carrier for activation, such as saline or water for injection, immediately before use.

Formulations of this invention suitable for oral administration may be developed as individual units, such as cachets, capsules, or tablets, each containing a predetermined amount of blocking agent or blocking agent and at least one of an antioxidant and an anticoagulant. The formulation may be presented as a powder or granules, as an oil in water liquid emulsions, or as a solution or suspension in an aqueous liquid or a non-aqueous liquid. The blocking agent or blocking agent and at least one of an antioxidant and an anticoagulant may also be given as a paste, electuary, or bolus.

Formulations of the invention may also be in the form of a tablet that can be taken on an acute or chronic basis to immediately or chronically treat injury. Tablets may be made by compression or molding, optionally with a blocking agent or a blocking agent and at least one of an antioxidant and an anticoagulant. Tablets made by compression may be prepared by compressing in a suitable machine the blocking agent or blocking agent and at least one of an antioxidant and an anticoagulant in a free flowing form such as granules or a powder, preferably mixed with a binder, inert diluent, binder, lubricating, surface active or dispersing agent. Molded tablets may be made in a suitable machine by molding a mixture of powdered compound moistened with an inert diluent that is in the liquid form. Tablets may be coated or scored, and may be formulated so as to provide controlled or slow release of the blocking agent or blocking agent and at least one of an antioxidant and anticoagulant therein.

Formulations for topical administration in the mouth include lozenges comprising the blocking agent or blocking agent and at least one of an antioxidant and an anticoagulant in a flavored form, such as with sucrose, acacia, or tragacanth and pastilles comprising the active ingredient in a foundation such as glycerin, gelatin, or sucrose and acacia. These formulations will be absorbed buccally of sublingually.

Rectal or vaginal formulations may be developed as suppositories with the standard carriers, such as polyethylene glycol or cocoa butter.

Preferred unit dosage formulations will be those containing an effective dose of the blocking agent or blocking agent and at least one of an antioxidant and an anticoagulant.

It should be understood that, in addition to the ingredients specifically described above, the formulations of this invention may include other agents known to one skilled in the art having for example flavoring agents may be used in formulations for oral agents or special kits with unique delivery systems may be employed for intravenous or intra arterial administration.

The blocking agent or blocking agent and at least one of an antioxidant and/or anticoagulant administered in the following doses: internal 1 pg/kg to 10 g/kg; topical from 0.0001% to 100%, formulated as compounds or mixtures for sustained or immediate delivery or release. Dosing regimens of the present invention include discrete doses of between 1 and 10 administrations per day, chronic multiple doses, acute single doses, as a bolus, or as a drip with constant IV or other infusion. The dosage of antioxidants and/or anticoagulants administered with the blocking agent will vary depending on the composition and combination of components used.

The dosage given to a patient will ultimately be the responsibility of the physician providing the invention. The dose used will depend on several factors, including the weight, age, gender of the patient, the severity of the injury, the route of administration, the acute of chronic nature of the injury and the precise disorder causing the injury. In addition, the route of administration will vary depending on many factors including the nature of the condition and the severity of the illness.

### EXAMPLES

The following Examples are provided as illustrative examples of methods and procedures used in the studies associated with the discovery of the injury induced calcium channel and the invention of compositions and methods for therapeutic treatment of the injury induced calcium channel.

### Example 1

### Method of Preparing Hippocampal Neurons in Culture

Primary hippocampal cultures were prepared by a modification of the method of Banker and Conan, as described by DeLorenzo et al. (incorporated herein in their entirety by reference). Banker, G. A. and W. M. Conan, Brain Res. 126:397-42, 1977; DeLorenzo, R. J., S. Pal, and S. Sombati, Proc. Natl. Acad. Sci. U.S.A. 95:14482-14487, 1998. Hippocampal neurons and other cells were dissected from 2-day postnatal Sprague-Dailey rats (Harlan, Frederick, Md.) and plated at a density of 2×10⁵ cells/chamber onto #1 cover glass chamber slides (Nunc, Naperville, Ill.) previously coated with 2 μl Matrigel Matrix (Becton Dickinson Labware, Bedford, Mass.). Cultures were maintained at 37° C. in a 5% CO₂/95% air atmosphere and fed three times weekly with neuronal feed containing MEM, 2 mM L-glutamine, 10 mM glucose, 5 μM/ml insulin, 100 μM/ml transferrin, 100 μM putrescine, 30 nM sodium selenite, 20 nM progesterone (ICN, Costa Mesa, Calif.), 1 mM sodium pyruvate, 0.1% ovalbumin (Fisher, Pittsburgh, Pa.), 20 ng/ml triiodothyronine (Calbiochem, La Jolla, Calif.), and 40 ng/ml corticosterone (ICN, Costa Mesa, Calif.). Since no astroglial feeder layer was present in these cultures, conditioned media was harvested from confluent astroglia cultures and added to neuronal feed (20% by volume). The first neuronal feeding included 5 μM cytosine arabinoside to ensure inhibition of non-neuronal growth. Microfluorometric analysis and electrophysiological studies were performed on neurons after 14-17 days in culture.

### Example 2

### Intracellular Calcium Measurements

Cell loading with indo-1 or Fura-FF:

To load hippocampal neurons with indo-1 or Fura FF, neuronal feed was removed and replaced with Recording Solution (145 mM NaCl, 2.5 mM KCl, 10 mM HEPES, 10 mM glucose, 2 mM CaCl₂, 1 mM MgCl₂, pH 7.3, osmolarity adjusted to 325 with sucrose) containing 1 μM indo-1 AM or 1 μM Fura-FF AM. Pal, S., Limbrick, D. D., and R. J. DeLorenzo. Cell Calcium 28: 181-193, 2000. Loading of cells was performed at 37° C. for 1 hour. Cells were then be washed three times and incubated for an additional 15 min to allow for cleavage of the indo-1 AM or Fura-FF to the free acids form by cellular esterases.

Microfluorometry:

[Ca²⁺]ᵢ was measured using the ratio program (Ca²⁺ imaging mode) of a confocal ACAS Ultima Interactive Laser Cytometer (Meridian Instruments, Okemos, Mich.) as described previously. Limbrick, D. D. J., S. B. Churn, S. Sombati, and R. J. DeLorenzo, Brain Res. 690:145-156, 1995; Wade, M. H., F. A. de, and M. K. Frame, Methods Biochem. Anal. 37:117-141, 1994. Briefly, a 100 mW, 360 nm line of argon laser was passed through an Olympus 100 UV/340 oil immersion objective (Lake Success, N.Y.) mounted on an Olympus IMT-2 inverted microscope (Lake Success, N.Y.). Emission wavelengths was passed through a 225 pm pinhole aperture before reaching the photo multiplier tubes (PMT). Photo currents from each PMT (set at 35%) were then measured by a computer-driven data acquisition system. The ratio of emitted wavelengths at 405 nm (indo-1-Calcium complex) and 485 nm (free indo-1) were monitored to indicate relative free calcium concentration. In experiments performed using the calcium indicator Fura-FF, optics were set for dual excitation imaging (340 and 380 nm wavelengths), and the emission wavelength collection were set to 510 m, as described previously. Pal, S., Limbrick, D. D., and R. J. DeLorenzo. Cell Calcium 28: 181-193, 2000. Hyre, K., S. D. Handran, S. M. Rothman, and M. P. Goldberg, J. Neuro. Sci. 17:6669-6677, 1997; Limbrick, D. D. J., S. B. Chum, S. Sombati, and R. J. DeLorenzo, Brain Res. 690:145-156, 1995.

Actual free [Ca²⁺]ᵢ values were generated by comparison of fluorescence ratio values to a Ca²⁺ calibration curve generated as described previously. Pal, S., Limbrick, D. D., and R. J. DeLorenzo. Cell Calcium 28: 181-193, 2000. Data were collected at 16 samples/point, scan strength was set at 10-25%, and a 1% Neutral Density Filter was used to prevent indo-1 photo bleaching. Distance between laser pulses (step size) was set between 0.5-3.0 μM so that single or multiple cells could be monitored simultaneously and analyzed individually. All experiments were performed in Recording Solution at 35-36 degrees C.

Calcium Calibration Curve:

In order to convert fluorescence ratios to Ca²⁺ concentration, an aqueous, in vitro calcium calibration curve was performed. Pal, S., Limbrick, D. D., and R. J. DeLorenzo. Cell Calcium 28: 181-193, 2000. Limbrick, D. D. J., S. B. Churn, S. Sombati, and R. J. DeLorenzo, Brain Res. 690:145-156, 1995; Wade, M. H., F. A. de, and M. K. Frame, methods Biochem. Anal. 37:117-141, 1994; Grynkiewicz, G., M. Poenie, and R. Y. Tsien, J. Biol. Chem. 260:3440-3450, 1985. Experimental parameters and instrumentation settings were identical to those used for cell culture experiments, and the buffer used was designed to approximate intracellular ion balance (100 mM KCl, 1 mM EGTA, 50 mM HEPES, pH 7.2). The curves were generated by measuring the change in fluorescence after adding known amounts of Ca²⁺ to buffer containing both indicators as described previously. Pal, S., Limbrick, D. D., and R. J. DeLorenzo. Cell Calcium 28: 181-193, 2000. Kd values were estimated using values reported in the literature. Pal, S., Limbrick, D. D., and R. J. DeLorenzo. Cell Calcium 28: 181-193, 2000. Grynkiewics, G., M. Poenie, and R. Y. Tsien, J. Biol. Chem. 260:3440-3450, 1985; Iatridou, H., E. Foukaraki, M. A. Kuhn, E. M. Marcus, R. P. Hangland, and H. E. Katerinopoulos, Cell Calcium 15:190-198, 1994. We defined healthy neurons as having resting [Ca²⁺]ᵢ, levels between 75-200 nM; neurons displaying initial baseline [Ca²⁺]ᵢ values higher than 200 nM were abnormal and not analyzed.

### Example 3

### Electrophysiology Experiments

Before each patch-clamp experiment, culture medium was replaced with recording solution (described above). Cultures were then transferred to the stage of an Olympus IX-70 inverted microscope equipped with phase-contrast optics (Lake Success, N.Y.). Experiments were performed on medium-to-large phase-bright hippocampal pyramidal neurons grown 13 to 17 days in vitro. Throughout each experiment, cultures were perfused continuously at 1 ml/min and maintained at 30 degrees C. using a heating stage. Patch electrodes (2-7 M ohms in resistance) were generated from borosilicate glass capillaries (WPI, Sarasota, Fla.) using a Brown-Flaming PC-80 puller (Sutter Instruments, Novato, Calif.). Electrophysiological recordings were conducted in the whole-cell patch-clamp configuration as described. Coulter, D. A., S. Sombati, and R. J. DeLorenzo, J. Neurophysiol. 68:362-373, 1992; Sombati, S., D. A. Coulter, and R. J. DeLorenzo, Brain Res. 566:316-319, 1991. Current-clamp experiments were conducted using the bridge mode of an Axoclamp 2A amplifier (Axon Instruments, Foster City, Calif.). Data were digitized and stored on videotape using a Neurocorder (Neurodata, Cygnus Technology, city, state) and played back for analysis a on a Dash IV chart recorder (Astro-Med, Warwick, R.I.). Voltage-clamp experiments were performed using an Axopatch 1D amplifier and pCLAMP 6.04 software (both from Axon Instruments). Series resistance compensation circuitry was used to cancel approximately 75-80% of the series resistance.

### Example 4

### Solutions

In all experiments, cultures were perfused first with control recording solution as described above. Glutamate (100-500 μM) was dissolved in recording solution and applied with 10 μM glycine for 10 minutes. Washout of the glutamate was performed with either control recording solution, recording solution plus dissolved drug, or Na⁺-free recording solution. In some experiments, drugs were applied in Na+-free recording solution. Na⁺-free recording solution was made by equimolar substitution of choline chloride or N-methyl-D-glutamine chloride for NaCl. All drugs were obtained from Sigma (St. Louis, Mo.) with the following exceptions: APV (RBI), CNQX (RBI), MCPG (Tocris, UK), SKF-96365 (Calbiochem, Calif.), and tetrodotoxin (Alomone Labs, Jeruselem, Israel). For patch clamp experiments, the intracellular/electrode solution contained (in mM): 140 K⁺ gluconate, 10 HEPES, 1 MgCl₂ pH 7.2. osmolarity adjusted to 310 mosm with sucrose.

### Example 5

Data Analysis

[Ca²⁺]ᵢ data were analyzed using Merlin (Life Science Resources) and SigmaPlot (Jandel Scientific, city, Calif.) software or as described previously. Pal, S., Limbrick, D. D., and R. J. DeLorenzo. Cell Calcium 28: 181-193, 2000. Voltage-clamp data were acquired with the use of CLAMPEX and analyzed using CLAMPFIT (both from pCLAMP 6.04, Axon Instruments). Current-voltage graphs were generated using Origin (Microcal Software, Northampton, Mass.). Statistical analyses were performed using Sigma Stat (Jandel Scientific).

Means of Data Analysis

Graphical and statistical analysis of, raw [Ca²⁺]ᵢ value and [Ca²⁺]ᵢ time courses were conducted using Sigma Plot and Sigma Stat software (Jandel Scientific, San Rafael, Calif.). Wade, M. H., F. A. de, and M. K. Frame, Methods Biochem. Anal. 37:117-141, 1994. In experiments monitoring several neurons simultaneously, the [Ca²⁺]ᵢ level in each cell were quantitated independently.

In some instances, [Ca²⁺]ᵢ values were normalized for comparison of [Ca²⁺]ₗ regulation kinetics. Normalization of the [Ca²⁺]ᵢ data (the average of multiple neuronal [Ca²⁺]ᵢ values was performed by division of all post-glutamate [Ca²⁺]ᵢ values by the peak [Ca²⁺]ᵢ value attained during the glutamate exposure. In order to analyze the effect of pharmacological inhibitors on the persistent influx of Ca²⁺, [Ca²⁺]ᵢ values recorded in the presence and absence of extracellular Ca²⁺ were normalized, and the normalized [Ca²⁺]ᵢ values recorded in the absence of extracellular Ca²⁺ were subtracted from the normalized [Ca²⁺]ᵢ values recorded in the presence of extracellular Ca²⁺. This subtraction process provides an estimate of the effect of extracellular Ca²⁺ on overall [Ca²⁺]ᵢ which served as an estimate of the influx component of the sustained elevations in [Ca²⁺]ᵢ. Conversely, the normalized [Ca²⁺]ᵢ curve recorded in the absence of extracellular Ca²⁺ served as an estimate of the ability of the neurons to extrude or sequester free intracellular Ca²⁺, since Ca²⁺ influx was be abolished under these conditions.

Those persons skilled in the art will appreciate that the present invention is susceptible to a broad utility and application. Many embodiments and adaptations of the present invention other than those herein described, as well as many variations, modifications will be apparent from or reasonable suggested by the present invention and the foregoing description thereof, without departing from the substance or scope of the present invention. Accordingly, while the present invention has been described herein in detail in exemplary embodiments, it is to be understood that this disclosure is only illustrative and exemplary of the present invention and is made merely for purposes of providing a full and enabling disclosure of the invention. The foregoing is not intended or to be construed to limit the present invention or otherwise to exclude any such other embodiments, adaptations, variations, modifications, the present invention being limited only by the claims appended hereto and the equivalents thereof.

1-33. (canceled)

34. A method of identifying an agent that protects neuronal cells from calcium influx into a neuronal cell through an injury induced calcium channel, the method comprising
contacting an injured neuronal cell with the agent; and
measuring the calcium influx through the injury induced calcium channel;
wherein a reduction in calcium influx through the injury induced calcium channel in said neuronal cell relative to a control neuronal cell which has not been contacted with the agent indicates that the agent can protect neuronal cells from calcium influx through the injury induced calcium channel.

35. The method of claim 34, wherein the injury induced calcium channel can cause neuronal cell death or damage.

36. The method of claim 34, wherein calcium influx through the injury induced calcium channel can be blocked by at least 100 μM of a gadolinium compound.

37. A method of protecting a neuronal cell from death or damage, comprising identifying an agent according to claim 34, and administering to a patient in need thereof an effective amount of agent to reduce calcium influx through the injury induced calcium channel thereby reducing neuronal cell death or damage relative to the absence of administering the agent.

38. A method of identifying an agent that protects neuronal cells from extended neuronal depolarization caused by calcium influx into a neuronal cell through an injury induced calcium channel, the method comprising
contacting an injured neuronal cell with the agent; and
measuring the extended neuronal depolarization in the neuronal cell;
wherein a reduction in extended neuronal depolarization in said neuronal cell relative to a control neuronal cell which has not been contacted with the agent indicates that the agent can protect neuronal cells from extended neuronal depolarization cause by calcium influx through the injury induced calcium channel.

39. The method of claim 38, wherein the injury induced calcium channel can cause neuronal cell death or damage.

40. The method of claim 38, wherein calcium influx through the injury induced calcium channel can be blocked by at least 100 μM of a gadolinium compound.

41. A method of protecting a neuronal cell from death or damage, comprising identifying an agent according to claim 38, and administering to a patient in need thereof an effective amount of agent to reduce calcium influx through the injury induced calcium channel thereby reducing neuronal cell death or damage relative to the absence of administering the agent.

42. A method of identifying an agent that protects neuronal cells from death or damage caused by calcium influx through an injury induced calcium channel in the neuronal cells, the method comprising
contacting injured neuronal cells with the agent; and
measuring the death or damage of the injured neuronal cells;
wherein a reduction in death or damage in the injured neuronal cells relative to a control neuronal cell which has not been contacted with the agent indicates that the agent can protect the neuronal cells from death or damage.

43. The method of claim 42, wherein calcium influx through the injury induced calcium channel can be blocked by at least 100 μM of a gadolinium compound.

44. A method of protecting a neuronal cell from death or damage, comprising identifying an agent according to claim 42, and administering to a patient in need thereof an effective amount of agent to reduce calcium influx through the injury induced calcium channel thereby reducing neuronal cell death or damage relative to the absence of administering the agent.
